(19) United States
(12) Patent Application Publication
(10) Pub. No.: US 2024/0033497 A1
(43) Pub. Date: Feb. 1, 2024

(54) MEDICAL DEVICE CONTROL SYSTEM, CONNECTOR AND COUPLING ASSEMBLY WITH PARAMETER INDICATION

(71) Applicant: Arjo IP Holding Aktiebolag, Malmö (SE)

(72) Inventors: Michael David Newton, San Antonio, TX (US); David John Cain, Houghton Regis (GB)

(73) Assignee: Arjo IP Holding Aktiebolag, Malmö (SE)

(21) Appl. No.: 18/267,922

(22) PCT Filed: Dec. 15, 2021
(86) PCT No.: PCT/SE2021/051264

§ 371 Date: Jun. 16, 2023

(30) Foreign Application Priority Data
Dec. 18, 2020 (SE) 2051491-5

(51) Int. Cl. A61M 39/16

(52) U.S. Cl. CPC A61M 39/16; A61B 90/70

(57) ABSTRACT

Present invention relates to a connector (330) for a coupling assembly (300, 600) for connecting a medical device (120) and a controller device (110) in a medical device control system (100). The controller device (110) is configured to control the operation of the medical device (120), the connector (330) having a connector body (331) being connectable to a connecting member (310) of the coupling assembly for forming a connection. The connector (330) comprises at least one parameter indicating element (390) provided on the connector body (331), said at least one parameter indicating element (390) being adjustable relative the connector body (331) for providing an indication of a use parameter of the medical device control system (100) to a user.

### TECHNICAL FIELD

The present invention relates to a connector for a coupling assembly, a coupling assembly, a medical device, a medical device control system and a method for providing a user an indication of a use parameter of the medical device control system.

### BACKGROUND

The field of medical devices includes products specifically intended for multiple uses across many patients and other products specifically intended for use with only one patient. The use of reprocessing techniques for such types of medical devices is becoming ever more commonplace in the medical device industry. As a result, there are many types of medical devices that are now being used multiple times by different patients but which were originally intended, designed, manufactured and sold for a single patient use.

This activity is both widely accepted within healthcare systems and specifically regulated. This is to ensure that there are controls in place to promote patient safety in a similar manner to those controls applicable to the original design and manufacture of the device. These regulatory controls are often focused on the cleaning process to ensure the resulting reprocessed device is suitably cleaned to avoid any risks between uses by different patients.

Another area covered by regulatory controls is the ensuring of that the reprocessing processes used on the device itself does not pose any hazard to the patient compared to the same device when it was originally manufactured. There are a number of advantages associated with the processing and reuse of devices when combined with a controlled cleaning or disinfection process such as reduced operational costs and a minimization of medical equipment waste. However, there can be some inherent limitations in what can be readily achieved with some devices and processes in terms of prolonging safe device reuse and ensuring suitable functionality.

There is also often less focus on the detection, monitoring and indication of exactly how many times a reprocessed device has been used or cleaned—particularly at the point of use in the clinical setting. Whilst reprocessors provide labelling of devices and can monitor cycles within the reprocessing supply chain, this opportunity is not always readily available to the end user where the device is in use.

The performance of some medical devices can change when they are operated beyond their original intended design lifetime or expected operational duration or when they are subjected to multiple reprocessing cycles. Whilst the reprocessing process requires validation and verification activities to be performed to define the number of cycles for a device, there is also always a limit to device lifetime. This is particularly the case where the manufacturer of the original device is also seeking ways to minimize the manufacturing and component costs of the device whilst ensuring ever increasing levels of performance and safety.

For example, there are changes that can occur in the dimensions and expansion rates of inflatable items such as compression garments and monitoring cuffs as a result of the effects of the natural fatigue of the flexible materials in normal use. Another example lies in components and materials found in medical devices that are subjected to changes in their characteristics as a result of the optical, thermal, radiation or chemical disinfection processes used in reprocessing.

All these factors can affect the reliability and durability of the reprocessed medical device. Addressing this aspect is clearly of benefit to the patient, user, manufacturer and reprocessor alike. It can help to ensure that the reprocessed device's operational lifetime is not exceeded and as a result prevent reduced performance or the increased risk of device failure. This can help to increasing patient safety and improving clinical outcomes when using the reprocessed devices.

There are a number of examples detailed in the prior art of the use of digital memory devices in many different types of medical devices for the purposes of recording aspects of operational or functional use (for example U.S. Pat. No. 6,884,255 and European patent EP2027825B2). However, there is often a need to provide this type of functionality in a lower cost and more robust manner that is consistent with device reprocessing. For example, some reprocessing methods use processes including irradiation or high temperatures that are not compatible with the use of digital memory devices, the invention described herein avoids these process limitations by utilizing alternative embodiments.

For some devices being reprocessed, there can be some inherent limitations and potential problems associated with some aspects of independent reprocessing. This relates to the natural wear and fatigue factors associated with continued and extended use. An example is in the field of pneumatic compression devices which are designed and rated by the manufacturer for an expected use or number of inflation cycles associated with the usage by a single patient. Whilst this type of device is normally designed to have a significant capability to operate for longer than intended—there is still an inherent limit before failure will occur. The materials involved are typically subject to fatigue effects caused by the expansion of the inflatable chamber(s) and also can be subject to changes in performance and deterioration as a result of certain reprocessing processes. This fatigue can result in device failure through leakages, the resulting occurrence of alarms or a reduction in the designed performance level.

In the light of the above, there is a need for a connector system which addresses and mitigates the risk for reprocessing and reuse of a worn out inflatable/deflatable article. There is further a need for a system which helps the user to identify a faulty or worn out connector or inflatable/deflatable article.

### SUMMARY

According to an aspect, a connector for a coupling assembly is provided. The connector is for connecting a medical device and a controller device in a medical device control system. The controller device is configured to control the operation of the medical device. The connector has a connector body. The connector body is connectable to a connecting member of the coupling assembly for forming a connection.

The connector comprises at least one parameter indicating element provided on the connector body. The at least one parameter indicating element is adjustable relative the connector body for providing an indication of a use parameter of the medical device control system to a user.

According to an aspect, a coupling assembly for connecting a medical device and a controller device in a medical device control system is provided. The controller device is configured to control the operation of the medical device. The coupling assembly comprises a connecting member and a connector according to the above described embodiment.

According to an aspect, a medical device system comprising a medical device, a medical device connection and a connector according to the above described embodiment. The medical device is connected to the connector by means of the article fluid connection.

According to an aspect, a medical device control system is provided. The medical device control system comprises a medical device and a controller device for controlling the operation of the medical device. The medical device control system further comprises a coupling assembly according to the above described embodiment.

According to an aspect, a medical device control system is provided. The medical device control system comprises a medical device and a controller device configured to control the operation of the medical device. The medical device control system further comprises a coupling assembly for connecting the medical device and the controller device.

The coupling assembly comprises a connector and a connecting member, the connector having a connector body connectable to the connecting member for forming a connection.

The connector comprises at least one parameter indicating element provided on the connector body, said at least one parameter indicating element being adjustable relative the connector body for providing an indication of a use parameter of the fluid pressure control system to a user.

According to an aspect a method of providing a user with an indication of a use parameter of a medical device control system according to the above embodiment is provided. The method comprises adjusting the position of the at least one parameter indicating element relative the connector body in response to a performed use event associated with the use parameter.

Further objects and features of the present invention will appear from the following detailed description of embodiments of the invention.

### BRIEF DESCRIPTION OF DRAWINGS

The invention will be described with reference to the accompanying drawings, in which:

FIG. 1 depicts an example of a medical device control system.

FIG. 2 depicts an example of a connector.

FIG. 3a-c depicts embodiments of a parameter indicating element according to the present invention.

FIG. 4 depicts a schematic system diagram of a medical device control system according to an embodiment of the present invention.

FIG. 5a-c depicts schematic graphs of the operation of a coupling assembly of a medical device control system according to an embodiment of the present invention.

FIG. 6 schematically depicts the operation of a connector according to an embodiment of the present invention.

FIG. 7a-d depicts aspects of a connector according to an embodiment of the present invention.

FIG. 8a-d depicts aspects of a connector according to an embodiment of the present invention.

FIG. 9 depicts an exploded view of a connector according to an embodiment of the present invention.

FIG. 10ab-b depicts a connector according to an embodiment of the present invention.

FIG. 11a-c depicts aspects of a connector and connection according to an embodiment of the present invention.

FIG. 12a-e depicts aspects a connector according to an embodiment of the present invention.

FIG. 13a-b depicts a connector according to an embodiment of the present invention.

### DETAILED DESCRIPTION

As will be eluded further, present inventors have realized that one of the most effective areas of a device to locate any means of monitoring and indication in a medical device control system is the connector. The connector is the area of a product that a user has the most direct association with in order to physically make use of the device. Hence any indication is most likely to be seen if located at this point, or the connector adjusted if that is the function. Also, it is an effective product location in terms of providing an indication to inform the user regarding the compatibility of a device with an item of equipment. In addition, at the end of use, a suitable color coding or marking can provide an effective indication of the next step upon device disconnection and allow the user to direct the product into the appropriate waste management channel.

A key aspect of the invention addresses a particular area of reliability where there is a medical system involving the combined operation of a durable piece of control/measurement equipment with an associated medical device. The medical device may be a disposable device which often is intended either specifically for single-patient use or for a defined period of usage or operational life. These devices are often reprocessed multiple times and hence there is a risk that they can be used beyond their original intended life. The durable equipment is typically used for many patients and over many years and therefore often has to operate with a variety of devices covering different models, versions and ages of accessories. The durable medical equipment that operates with these reprocessed disposable devices often does so in a manner that is the same for a reprocessed device as it does for new device. This is because the durable equipment is often not able to sense the degree of prior usage or the reprocessing/cleaning steps that the disposable device has been subjected to. With the use of intelligent measurement and control techniques within the durable equipment and a means of identification present in the disposable device some of these limitations can be addressed. The ability to detect the prior usage and automatically adjust aspects of device operation is therefore beneficial.

Further, in certain situations, it is advantageous if the reprocessing of an individual device can be undertaken by multiple organizations during its lifetime with the reprocessing history of the device spanning multiple organizations. The device is therefore effectively managed by differing organizations over its operational life. This can present a problem since this usage information is not typically shared between competing organizations or the data storage formats are proprietary and not readily shareable. The approach detailed herein allows this usage information to be readily shared as it is carried on the device itself. Further the usage information is provided in a more shareable format as a means of recording a parameter or record of its use, described herein as a use parameter

Many reprocessing companies reprocess various medical devices such as compression garments. As a result, it is important that the actual usage is monitored in some manner and the invention described herein provides a means to achieve this. This allows for preventative steps and mitigations to be included in the operation of the device during use as well as during subsequent reprocessing processes. This can therefore lead to improvements in the quality of reprocessing activities as well as the actual device operation with a patient.

Many original equipment manufacturers (OEM's) now offer their own reprocessing operations so that they can provide both new and re-processed devices to customers. This approach ensures that the reprocessing of devices can be validated consistent within the limitations of their original design and construction. In the case where the operation of the medical product is a combination of durable equipment and a disposable item, the OEM is also able to look at both the device to be reprocessed and also the interaction and detailed operation of the associated durable equipment.

This aspect is not readily feasible for independent reprocessors as they not always fully aware of all the complex details of the original product performance in terms of their detailed interaction with associated durable equipment. This information can involve a range of technical aspects such as materials used, weld strengths, expansion characteristics, tolerances, proprietary and otherwise internal or hidden features, functional risk mitigations and alarm thresholds as well as test results of the original intended life.

As a result, the effectiveness of any independent reprocessing is often limited in terms of the number of cycles, performance or increased life that can be achieved.

An individual independent reprocessor can add additional controls such as additional labelling to a reprocessed device to provide some benefits but this can be easily damaged or lost between reprocessing cycles and this is not integrated into the product operation. It does not provide any information on usage to the other items of equipment necessary for use of a device—e.g. as in the case of a compression pump and garment.

Alternatively, this type of capability can be included in the initial design and manufacture of the device and so can avoid introducing additional unnecessary costs to the device. It can also be added post-manufacture to the device, for example by an independent device reprocessor. The present invention provides a means where a device connector can be easily configured in a low cost manner at any point in its lifecycle to add usage monitoring and indication features easily. The manufacturer can provide a provision for a secondary part that can be subsequently fitted by a reprocessing partner. This advantageously allows for a reduction in the cost of newly made parts and the cost of the reprocessing feature is only added to those devices actually reprocessed. This allows for savings in use by healthcare facilities as well as a reduction in material usage and waste creation.

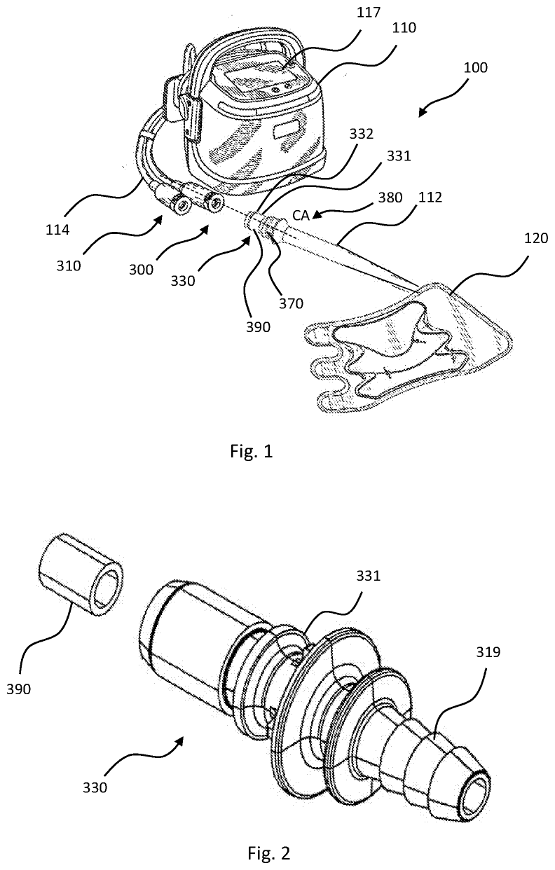

FIG. 1 depicts a medical device control system wherein a connector according to the present invention may be implemented.

The medical device control system 100 comprises a medical device 120. The medical device control system 100 further comprises a controller device for controlling the operation of the medical device 120.

The medical device control system further comprises a coupling assembly 300 for connecting the medical device 120 and the controller device 120. The coupling assembly comprises a connector 330 and a connecting part 310.

The connector 330 has a connector body 331. The connector body is connectable to the connecting member 310 for forming a connection. The connection may be formed through said connector 330 and connecting member 310.

The connector 330 may be connectable to the connecting member 310 to form various electrical, fluid or optical connection. In one embodiment, the connector 330 is connectable to the connecting member 310 to form a plurality of an electrical, fluid or optical connection.

The medical device 120 may be any one of an inflatable/deflatable article, a measuring device and a disposable medical device.

In the embodiment shown in FIG. 1, the medical device is an inflatable/deflatable article and the controller device is pump. Accordingly, the coupling assembly forms a fluid connection between the inflatable article and the controller device.

Accordingly, the medical control system may be fluid pressure control system wherein a connector according to the present invention may be implemented. The fluid pressure control system 100 may be a gas pressure control system such as a pneumatic control system or may be based on any type of suitable fluid for the application with inflatable/deflatable articles.

The medical device control system 100 comprises the medical device 120 and a controlling device 110. The controlling device 110 is configured to control the operation of the medical device 110.

The controlling device may comprise a control unit (not shown in FIG. 1). In one embodiment, the control unit is operatively connected to a pump of the controlling device 110 for controlling said pump.

In one embodiment, the pump may be a pneumatic pump. The pump may be arranged to control fluid flow to and from the inflatable/deflatable article. Accordingly, the pump may be arranged to inflate or deflate the inflatable/deflatable article.

The medical device control system 100 comprises the coupling assembly 300 for connecting the medical device 120 and the controlling device 110. The coupling assembly 300 comprises a connector 330 and a connecting member 310.

The connector 330 has a connector body 331. The connector body 331 is connectable to the connecting member 310 for forming a connection through the connector 330 and the connecting member 310. In one embodiment, the connector 330 and the connecting member 310 are connectable to form a fluid pathway through the connector 330.

The connection through the connector 330 and the connecting member 310 may be formed by means of insertion of the connector body 331 or a part of the connector body 331 into the connecting member 310. Thus, the connector body 331 may have a distal part 332 for coming into engagement with the connecting member 310.

The connector body 331 is movable inside the connecting member 310 along a connection axis CA. The connection axis CA extends distally from the connector 330, The distal part 332 is movable inside the connecting member 310 along the connection axis CA. Preferably, the distal part 332 and the connecting member 310 are adapted to sealingly engage when the connector body 331 is in a coupled position.

The connector body 331 may be movable from a non-inserted position to the non-coupled position. In the non-coupled position, the connector body 331 may have at least come into contact with the connecting member 310. A non-coupled position herein refers to a position of the connector body 331 inside the connecting member 310 wherein the coupling assembly does not provide a connection through the connecting member 310 and the connector 330. Correspondingly, a coupled position herein refers to a position of the connector body 331 inside the connecting member 310 wherein a connection through the connecting member 310 and the connector 330 is achieved.

In one embodiment, substantially the entire length of the distal part 332 may be inserted into the connecting member 310 when the connector body 331 is in the coupled position.

Further referencing FIG. 1, the medical device control system 100 comprises a medical device connection 112. The medical device 120 may be connected to the connector 330 by means of said medical device connection 112.

Thus, a medical device system may comprise the medical device 120, the medical device connection 112 and the connector 330. The medical device 120 being connected to the connector 330 by means of the medical device connection 112.

The medical device control system 100 may comprise a controller device connection 114. The controller device 110 may be connected to the connecting member 310 by means of said controller device connection 114.

In one embodiment, wherein the medical control system is a fluid pressure control system. The connector 330 may be fluidly connected to the medical device 120 by means of a device fluid connection (i.e. medical device connection 112). The article fluid connection may be a tube or a hose. Correspondingly, the connecting member 310 may be fluidly connected to the controller device by means of a controller device fluid connection (i.e. controller device connection 114). The controller device fluid connection 114 may be a tube or a hose.

According to an embodiment, the coupling assembly 300 may further comprise a mechanical latch 370. The mechanical latch 370 is arranged to secure the distal part 331 is in the coupled position. The coupled position may thus be a latched position of the distal part.

The mechanical latch 370 may be a manually operated mechanical latch adapted to be engaged by a user to secure the connector body 331 when said connector body 321 is in the coupled position, i.e. is in engagement with the connecting member 310.

Alternatively, the mechanical latch 370 is adapted to resiliently engage to secure the connector body 331 when said connector body 331 is in the coupled position.

Preferably, the mechanical latch 370 comprises a locking member provided on the connecting member 310 or the connector 330 and a retention member provided on the other of the connecting member 310 or the connector 330. When the connector body 331 is in the coupled position, the retention member is arranged to engage the retention member, whereby the mechanical latch 370 is secured relative the connecting member 310.

Mechanical latches are well-known in the prior art and will not be described in further detail.

Referencing FIG. 2, a connector 330 in which the functionality of the present invention may be implemented is depicted. The connector 330 comprises at least one parameter indicating element 390.

According to the present invention, the parameter indicating element 390 may be provided on the connector body 331. The at least one parameter indicating element 390 is adjustable relative the connector body 331 for providing an indication of a use parameter of the medical device control system to a user.

A beneficial aspect of the invention is that the same physical features, i.e. components of the control system, can be used within one or more healthcare facilities for totally different purposes. For example, this could include the recording and indication of the number of inspections of a medical device or the recording and indication of the number of days usage of a device. Another aspect involves when cleaning is performed between individual patient usages, the same recording and indicator means can be used to track these activities within a facility, so it can provide a device-mounted hygiene, disinfection or washing activity recording means. The same indicator can also be used internally in the facility to support healthcare process quality activities though allowing a user-implementation means of marking devices themselves and their use with different patient risk types.

A further application of the invention lies in the field of medical device waste management and its reduction within a healthcare system. This aspect is becoming ever more important as facilities implement workflow practices to achieve environmental management and cost reduction activities.

Since each facility has its own waste management process, it is helpful if products are clearly marked to the end user to allow them to follow the prescribed disposal process at the end of a patient usage.

Thus, the above described connector allows for the connector itself to provide the functionality of indicating service parameters to a user. Thereby, a safer and reliable medical device control system may be achieved.

The use parameter may be considered as a parameter relating to the use of the medical device control system or a component of the medical device control system.

The use parameter may be any one of the group: cleaning of any one of the components of the medical device control system 100, disinfection of any one of the components of the medical device control system 100, sterilization of any one of the components of the medical device control system 100, unpackaging of any one of the components of the medical device control system 100, assignment of the medical device 120 to a patient, reprocessing of the medical device 120, uses of any one of the components of the medical device control system 100 or end of use of any one of the components of the medical device control system 100.

Components herein refers to any one of the parts of the medical device control system. Accordingly, said component may be the controller device 110, the medical device 120, the connector 330, the connecting member 310 or any other of the components of the medical device control system.

As the skilled person realizes, movement of the parameter indicating element may be suitable to provide indications concerning additional use parameters of relevance.

The at least one parameter indicating element 390 may be adjustable relative the connector body 331 such that the position of the at least one parameter indicating element 390 is adjustable to provide a user with a use value associated with the use parameter. The use parameter may for example be any one of the above described use parameters.

Preferably, the use value may relate to a performed use event of the use parameter. Use event herein refers to a performed act or event occurring during use and/or service life of the components or entire medical device control system 100.

In one embodiment, the performed use event is any one of the group: performed cleaning of any one of the components of the medical device control system 100, performed disinfection of any one of the components of the medical device control system 100, performed sterilization of any one of the components of the medical device control system 100, performed unpackaging of any one of the components of the medical device control system 100, performed assignment of the medical device 120 to a patient, performed reprocessing of the medical device 120, performed uses of any one of the components of the medical device control system 100 or established end of use of any one of the components of the medical device control system 100.

In one embodiment, the use value indicates a counter value, i.e. a number of performed acts. In one, embodiment, the use value indicates a binary value, i.e. an indication of whether an act has been performed or not.

The parameter indicating element 390 may be adapted to provide a user an indication in response to the counter value exceeding a predefined counter value.

In one embodiment, the parameter indicating element 390 may be adapted to provide a user an indication in response to the counter value exceeding one of a number of predefined counter values or threshold. These predefined counter values or thresholds being present or stored in the control system 110 or controller, for example in software.

Further, the at least one parameter indicating element 390 may be adjustable between a plurality of discrete positions relative the connector body 331. In other words, the parameter indicating element 390 may be adjustable between a plurality of distinguishable positions relative the connector body 331.

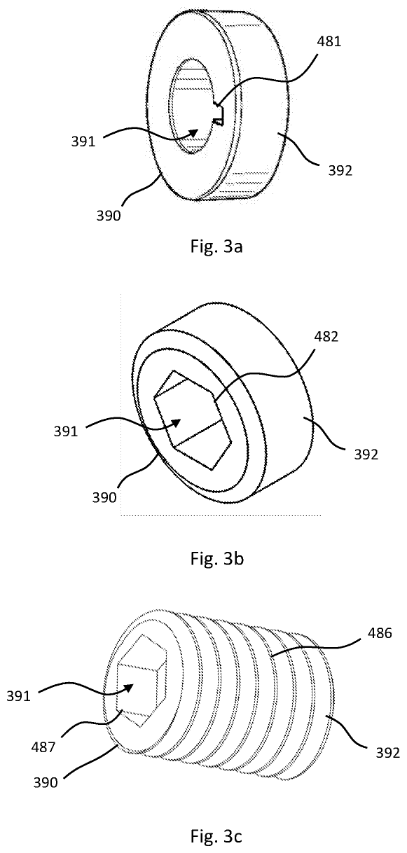

As will be further described with reference to FIGS. 3 to 13, the at least one parameter indicating element 390 may only be adjustable in one direction. This prevents tampering and accidental indications due to the user not being able adjust the parameter indicating element backwards when the use parameter is related to a count of a use event. The at least one parameter indicating element 390 may be unidirectionally adjustable.

Alternatively, the parameter indicating element may be adjustable in multiple directions. For example the parameter indicating element may be adjustable along the connector body in two directions (i.e. forwards and backwards).

As will be further described with FIGS. 3 to 13, the use value may be adjusted automatically or manually.

In one embodiment which provides automatic adjustment, the position of the at least one parameter indicating element 390 is adapted to be automatically adjusted in response to a change in the use value. Thereby, the user does not have to rely on remembering to manually adjust the parameter indicating element as soon as a notable change in the use parameter has occurred.

In one embodiment which provides manual adjustment, the position of one of the at least one parameter indicating element 390 is manually adjustable.

In one embodiment, the connector 330 may comprise both automatically and manually adjustable parameter indicating elements 390. Thereby, redundancy may be introduced in the indication of the use parameter.

In one embodiment, the position of the at least one parameter indicating element 390 may be detectable by means of a sensing arrangement of the medical device control system 100 such that an indication is provided based on a detected position obtained by the sensing arrangement (not shown in FIG. 1).

Again referencing FIG. 1, the medical device control system 100 may further comprise an indicating device 117. The indicating device 117 is operatively connected to the controller device 110, i.e. the control unit of said controller device 110. The indicating device 117 is configured to provide an indication of a use parameter to a user based on the detected position of the parameter indicating element obtained by the sensing arrangement, i.e. based on input from the sensing arrangement. Thereby, the user may be provided with an indication of a use parameter in a simple and clear manner.

The indicating device 117 may be provided on the controller device 110. Accordingly, the indicating device 117 may be mounted to the casing of the controller device 110. Thus, the user may be provided an indication while operating the medical device control system and in particular the controller device 110.

In one embodiment, the indicating device 117 may be a display unit, such as an LCD-display.

The parameter indicating element 390 may be arranged inside the connector body 331. This prevents tampering and incorrect adjustment of the parameter indicating element 390, thereby increasing the accuracy of the indication provided to the user. Thus certain aspects associated with the connector of a medical device are intentionally not user accessible such as the position of an parameter indicating element within the connector.

Accordingly, the connector body 331 may comprise an aperture for receiving said parameter indicating element 390. The connector body 331 may be a connector barrel, whereby said aperture may be the inner space of said barrel.

In one embodiment, the connector body may be fitted in a connection line of the connector body 331. The connection line may be formed as a fluid passage.

FIG. 3a-c depicts embodiments of parameter indicating elements 390 which allows for adjustments inside the connector body 331. The parameter indicating element 390 may be provided with retention means for allowing adjustment by means of rotation of the parameter indicating element 390 relative the connector body 331.

Thus the retention means may be arranged to allow for translational adjustment by means of rotation of the parameter indicating element 390.

In one embodiment, the parameter indicating element 390 may be adjustable along a distance extending along the connector body 331. Accordingly, the parameter indicating element 390 may be adjustable along the connection axis (depicted in FIG. 1).

In the case of the adjustment to the parameter indicating element position, this adjustment can utilize a simple notch or keyway (FIG. 3a) provided in the parameter indicating element to allow a tool to rotate and/or press the component into a new adjusted position. This can be achieved either by hand or via a mechanized method. Alternatively, one of many other types of features such as a slot, non-symmetrical internal shape or a hexagon hole (FIG. 3b) can be provided in the component to allow for engagement to achieve the adjusted position.

The external face of the component can have a screw thread or similar prominent feature (FIG. 3c) to provide both retention and grip as well as a means of traction within the connector body and hence facilitate the change in position during the adjustment process.

Referencing FIG. 3a, the retention means may comprise a keyway 481. The parameter indicating element 390 comprises a though-hole 391, the keyway 481 may be formed as a notch in said through-hole 391.

Referencing FIG. 3b, the through-hole 391 of the parameter indicating element 390 may be arranged to provide a grip for a tool to allow adjustment of the parameter indicating element 390. The through-hole 391 may have a non-symmetrical cross-section and/or a non-circular cross-section.

Referencing FIG. 3c, the parameter indicating element 390 may comprise a threaded outer surface 486 for adjustably engaging the aperture of the connector body. Accordingly, the aperture of the connector body may be provided with a corresponding threaded inner surface for adjustably receiving the outer surface 486 of the parameter indicating element 390.

Preferably, only the connector body 331 is arranged to adjustably receive the parameter indicating element and not vice versa. Often, the connector body 331 is in a polymeric material, thus allowing for easier forming of guiding means such as threads or guiding channels compared to providing it on the parameter indicating element.

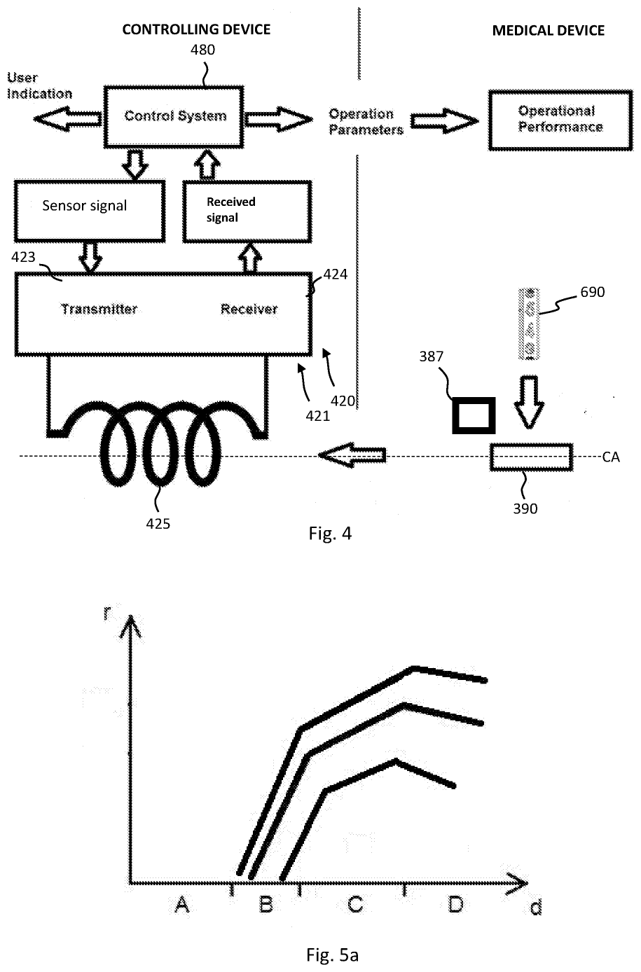

Turning to FIG. 4, an embodiment wherein the indication is provided to the user by means of the position of the parameter indicating element being detectable by means of a sensing arrangement is described in further detail.

The medical device control system may comprise the sensing arrangement 420. The sensing arrangement 420 may be operatively connected to the control unit 480 of the controller device. The sensing arrangement 420 is configured to detect the position of the parameter indicating element 390. The indication of the use parameter is provided to a user based on the detected position of the parameter indicating element 390 obtained by the sensing arrangement 420.

The sensing arrangement 421 is thus configured to detect the position of the parameter indicating element 390 when the connector body is in engagement with the connecting member, i.e. connected to the connecting member.

For example, the position of the parameter indicating element 390 within the connector body 330 may be capable of being measured in terms of a response. The parameter indicating element 390 may thus be mounted within the connector body 331. The connector being capable of being detected by the controller device, i.e. by the sensing arrangement. The controller device, i.e. the sensing arrangement being able to sense both the presence of the parameter indicating element in terms of the magnitude of response and the detailed response associated with a specific position of the parameter indicating element such that it can be differentiated from one of many positions. The same parameter indicating element is capable of being adjusted in position to provide a means of recording use values such as ‘counting’ or detailing of different usages, cycles or events, such as the usage or reprocessing of the compression garment itself. The adjustment range available to the positions of the parameter indicating element is typically in the range of 5 mm.

In one embodiment, the parameter indicating element 390 mainly comprises any one of ferrite, brass or steel. Thus, the parameter indicating element 390 may be detectable by means of mainly comprising a material detectable by means of the sensing arrangement.

The medical device control system may thus comprise a position indicating arrangement. The position indicating arrangement in turn comprises the parameter indicating element 390 and the sensing arrangement.

The position indicating arrangement 380 is configured to generate a position signal to the control unit 480. The position signal is indicative of the position of the parameter indicating element 390 relative the connector body.

To allow for precise detection, preferably, the parameter indicating element 390 may have a length extending along the connection axis of more than 2 mm.

In one embodiment, the parameter indicating element 390 may have a generally cylindrical or toroidal shape. Advantageously, the parameter indicating element 390 has an outer dimension (i.e. maximum width or height orthogonal to the connection axis) of between 5 and 10 mm and preferably between 6 and 8 mm. The parameter indicating element 390 may have an inner dimension (i.e. an inner diameter) of preferably less than 6 mm and more preferably greater than 4 mm. The parameter indicating element 390 may have a length extending along the connection axis of between 1 and 10 mm and more preferably between 2 and 9 mm.

A number of further alternative embodiments of parameter indicating elements 390 exist that are within the scope of the invention and should be obvious to anyone skilled in the art of position sensing and object detection.

The position signal may be based on a measured value obtained by the sensing arrangement 420. Accordingly, the sensing arrangement 420 may be configured to obtain a measured value based on the position of the parameter indicating element 390.

In one embodiment, the sensing arrangement may be an induction based sensing arrangement such as a radio based sensing arrangement. In alternative embodiment, the sensing arrangement may be an optically based sensing arrangement, whereby the parameter indicating element is detectable by means of optical sensors.

Thus, the position of the parameter indicating element 390 may be detected through the use of an electronic circuit comprised in the sensing arrangement. In one embodiment, the electronic circuit is a radio-based electronic circuit operating between and 300 kHz.

The electronic circuit may be physically located in the connecting member and/or the controller device (such as a pump). The electronic circuit may be able to sense a characteristic response from the identification device 390 in the connector body.

As depicted in FIG. 4, the sensing arrangement 420 may comprise at least one sensor unit 421.

The use of non-contact based sensing is particularly advantageous as it avoids a number of issues associated with potential alternative embodiments that use a physical contact means such as problems associated with the buildup of debris/material on contacts, regulatory concerns regarding exposed electrical contacts and physical damage to the alignment of a contact.

The sensor unit 421 may comprise a transmitter 423 and a receiver 424. The transmitter 423 is configured to generate a sensor signal. The sensor signal is received by the receiver 424. The position signal is based on the signal received by said receiver 424. The signal received by the receiver 424 may be considered the measured value obtained by the sensing arrangement.

Preferably, the sensing arrangement may be located in the connecting member or coupling 310 as well as the controller device 110.

In one embodiment, the sensor unit 421 may be provided on the connecting member 310. In one embodiment, the sensor unit 421 may be arranged externally from the connecting member 310.

Thus, the sensor unit 421 may be provided distally to the controller such as on the connecting member or alternatively may be located more proximally such as on the body (casing) of the controller device 110.

Preferably, the sensor unit 421 is arranged externally from the connecting member 310, this allows for mounting of the sensor unit 421 to the controller device 110. Thereby, the electronics of the system may be kept together on a single PCB which is advantageous both from a cost and complexity standpoint. Further, this allows for a connecting member without costly electronic components which makes it easier and cheaper to replace.

As will be described in further detail below, the sensing arrangement as a whole may be arranged on both the controller device and connecting member.

The transmitter 423 is configured to generate a sensor field along the connection axis CA. The position signal may be generated based on the position of the parameter indicating element 390 obtained by the receiver 424.

As depicted in FIG. 4, the sensing arrangement may be an induction based sensing arrangement. Thus, the sensor device may be an induction sensor. Preferably, the sensing arrangement may be a radio-based sensing arrangement.

The sensing arrangement 420 may further comprise a sensor coil 425. The sensor coil 425 may be operatively connected to the transmitter 423 and the receiver 424. In one embodiment, the sensor coil 425 may be arranged to be coaxial to the connection axis CA. The sensor coil may be configured to couple the transmitter 423 and the receiver 424.

Said sensor coil may be configured to generate an electromagnetic field extending along the connection axis CA, whereby the parameter indicating element 390 is detectable inside said electromagnetic field. The parameter indicating element 390 causes a change in the received signal compared to the sensor signal indicative of the position and/or movement of the parameter indicating element inside said electromagnetic field.

Accordingly, the configuration of the sensor coil 425 may be chosen such that the electromagnetic field extends along said connection axis CA.

The type and magnitude of the signal received by the receiver is based on the type, size and distance of the parameter indicating element in the connector relative the sensing coil.

Preferably, the sensor coil 425 is provided on the connecting member 310. In one embodiment, the sensor coil 425 may be provided inside the connecting member 310.

Having the sensor coil 425 provided on the connecting member 310 allows for easy service and potential replacement of the coil.

In an alternative embodiment, the sensor coil 425 may be provided in the controller device connection 114.

In one embodiment, the sensing arrangement is only able to detect the position of the parameter indicating element 390 when the connector is in close proximity to the connecting member.

As previously described, the parameter indicating element 390 may preferably be made of a material selected from a group consisting of a ferrite material and a brass material. Certain grades of steel and brass may also be used due to being able to produce a similar effect.

The material (for example ferrite) in the parameter indicating element 390 forms a variable permeability core to the sensor coil 425. Thereby, the coil inductance is modified. This change in inductance can be detected by means of electrical circuitry in the control unit 480 as a phase change in the sensor coil current resulting from the applied waveform signal and also as an amplitude change to the current flowing in the sensor coil 425. This change is a time-varying function of the position of the parameter indicating element relative the sensing arrangement (i.e. the sensor coil 425).

The control unit 480 may be configured to continually monitor the position signal and compare the measured value with reference measured values stored in the memory of said control unit 480.

Changes between the sensor signal and the received signal are indicative of the position or movement of the parameter indicating element 390. Such changes may be in the form a changed amplitude and phase in the received compared to the sensor signal. The received signal may be configured to be processed by the control unit 480. Said control unit 480 may be further configured to measure the phase change between the sensor signal and the received signal. The frequency of the received signal compared to the sensor signal may be largely unchanged.

In one embodiment, a single coil may be used to transfer the sensor signal between the transmitter and receiver. Accordingly, the transmitter 423 and receiver 424 may be in electrical connection with the sensor coil 425. The electrical connection is, in preferred embodiments, arranged such that the transmitter 423 and receiver 424 are in electrical conductive connection through the sensor coil 425. In one embodiment, the sensor coil 425 may be mounted in the connecting member. In one embodiment, may be mounted in the casing of the controller device.

Other embodiments within the scope of the invention include the use of a split coil with independent connections/windings where the transmit and receive signals are separate. Thus the sensing arrangement may comprise a receiver coil and a transmitter coil, whereby the received signal is separate from the sensor signal, i.e. the signal transmitted from the transmitter coil.

Alternatively, it is also possible to use separate transmit and receive coil arrangements where the two coils are always used for different purposes.

In one embodiment, wherein the connector provides a fluid connection, the sensor coil 425 is arranged to allow for fluid flow through a central axis of said sensor coil 425. The central axis of the sensor coil 425 may be substantially aligned with the connection axis CA.

In one embodiment, the sensor coil 425 may in the form of a ‘Brooks’ coil, i.e. being dimensioned according to well established ‘Brooks coil’ relative dimensions to allow for manufacturing efficiencies in coil winding and maximizing the resulting inductance provided by the wire used in the coil. This dimensional requirement is extended such that the sensor coil 425 may have a length of 5 mm in the direction of the connection axis CA. This allows ensures that the majority of the resulting electromagnetic field can be entered and exited during the insertion process, Hence the transition of the identification device 390 can provide the necessary response signals upon entering and transition through the coil as shown in FIG. 4.

The aforementioned coil dimensioning helps to optimize operation in use, improve coupling and reduces the physical size requirements whilst ensuring maximum coil sensitivity to the introduced parameter indicating element material.

This optimal dimensioning involves ensuring the ratio of the inner coil diameter of the sensor coil 425, which may form the path for the distal part of the connector and therefore the fluid flow, to the coil length being at least 2, and preferably the ratio of sensor coil 425 outer diameter to parameter indicating element length being 5.

In one embodiment, the sensor coil 425 has an inductance of 400-500 uH, preferably 446 uH when no parameter indicating element 390 is present in the coil. The inductance changing in response to the presence of the parameter indicating element 390.

In one embodiment, the system as whole (formed from coil, associated controller device circuitry and parameter indicating element) is tuned to be resonant between 80 kHz and 300 kHz and preferably around 125 kHz. Accordingly, the controller device, the sensing arrangement 420 and the parameter indicating element 390 may be tuned to operate between 80 kHz and 300 kHz and preferably around 125 kHz.

Further referencing FIG. 4, the connector 330 may comprise a data storage device 387. The data storage device 387 may contain data associated with the medical device 120 or the controller device 110.

In one embodiment, the data storage device 387 may be an RFID-device.

The controller device may thus further comprise data storage receiver means for reading the data contained on the data storage device 387.

In one embodiment, the data storage device 387 may contain data associated with a use parameter of the medical device control system. In one embodiment, the data is associated with a use parameter of the medical device. In one embodiment, the data is associated with a use parameter of the controller device.

The data comprised in the data storage device 387 may serve as an adjunct or extension to the position of the parameter indicating element. Accordingly, the data storage device 387 be an additional carrier of data relating to the use parameter.

In one embodiment, the at least one parameter indicating element 390 is in the form of an identification component. As is known in the prior art, an identification component is adapted to generate a characteristic response which serves to allow for identification of a particular component.

Accordingly, the identification component is adapted to generate a characteristic response associated with any one of the components of the medical device control system. The characteristic response may be detectable by means of the sensing arrangement 420 for identifying said any one of the components of the medical device control system.

Preferably said identification component is for identifying the type of medical device which is connected to the controller device. The identification component may thus be associated with said medical device.

The identification component may be adapted to generate said characteristic response by means of being in any one of a characteristic material, shape or dimension.

Thus, the system may provide the functionality of the indication of the use parameter and the identification of a particular component in a single component which reduces the complexity and allows for easier retro-fitting of the functionality to existing connectors.

The sensing arrangement 420 may thus be configured to detect the characteristic response generated by the identification component associated with any one of the components of the medical device control system for identifying said any one of the components of the medical device control system.

The control unit may be configured to compare said characteristic response with a set of predefined characteristic responses associated with a set of components in order to identify the component of the medical device control system associated with the identification component.

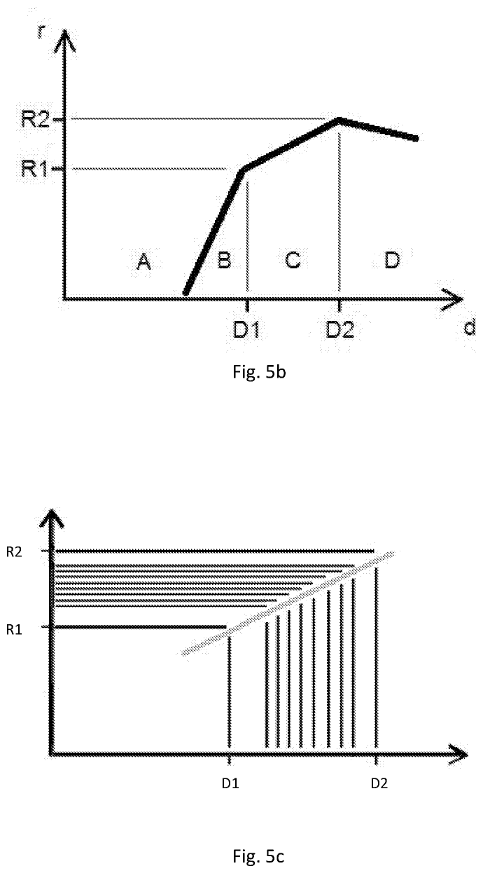

FIGS. 5a-b shows graphically a general response characteristic in a medical device control system. In this case it is a general response characteristic of a pump identification and measurement system including sensing the parameter indicating element at different locations in the pump connector. The amplitude of the identification response ‘r’ (shown on the Y axis), i.e. the signal received by the receiver, is plotted against the position ‘d’ of the parameter indicating element within the connector barrel (shown on the X axis). The below also applies for cases where the parameter indicating element is in the form of an identification component. Said identification component may be sensed in an identical manner.

It can be seen that there are a number of distinctly different areas of this response characteristic. There is an initial lack of response when the component is in a first range of positions (labelled A), due to the parameter indicating element lying largely outside of the sensing range. A second range of positions show a rapid change of response (labelled B) for a correspondingly small change in position, a third range of positions where there is a more gradual change in response to positional changes (labelled C) and a fourth range where the response becomes notably different for further increases in position (labelled D). The intended operation of the invention is in the area of FIG. 5a labelled C as this is associated with maximum sensitivity.

In normal operation, the minimum limit of position of D1 results in a maximum response of R1 and the maximum limit of position D2 results in a maximum response of R2. Thus, the received sensor signal reaches a maximum value in position D2 and a minimum in position D1.

There are multiple positions available located between lower positional limit D1 and the upper positional limit D2 that result in multiple individual responses. A lower limit R1 and upper limit R2 are used to define the normal range of operating parameters for a given garment/device type. The lower positional limit may be a first end position of the parameter indicating element and the upper positional limit may be a second end position of the parameter indicating element.

Accordingly, each of the discrete positions of the parameter indicating elements may be associated with a corresponding response, i.e. received signal.

The physical distance associated between the D1 and D2 points is within the range of >2 mm and <7 mm.

Since the component length itself can be in the range 2 mm to 7.5 mm, in some cases the component can travel a distance that is longer than its own length within the connector. This allows for a suitably accurate and high resolution of measurement to be achieved and is shown as individual response ranges for any given parameter indicating element in FIG. 5a and also values in the range R1 to R2 in FIG. 5c for a specific parameter indicating element.

FIG. 5c details the response in the FIG. 5a area of the graph labelled ‘C’ in more detail. The positional distance D1, i.e. the lower positional limit D1, results in a response R1, the response is able to be progressively increased as the position changes. In FIG. 5a-5c this is shown with a positive gradient in ‘r’ however it is also within the scope of the invention that a negative gradient can be achieved thought selection of materials, circuitry, construction and position.

As can be seen from FIG. 5c, a number of discrete response measurements, i.e. received signals, are possible. The received signals may be obtained by the control unit 480 (depicted in FIG. 4), whereby the control unit 480 is configured to determine the position of the parameter indicating element. The user is provided the indication of the use parameter based on the determined position, this may be performed by means of the indicating device.

In one embodiment, the control unit 480 (depicted in FIG. 4) may be configured to control the operation of the medical device based on the position, i.e. determined position, of the parameter indicating element. For example, this allows for the operation of the system to optimize its functional parameters based on the degree of prior usage of the medical device even if this usage of the reprocessed device was not with the currently connected controller device. The operation of the system can even be optionally inhibited to prevent operation if the connector is sensed as having reached its end of use as a result of the detected position of the parameter indicating element. Thus, a connector which allows tracking of the use parameters of the medical device regardless of which connecting device it is connected to and subsequent control of the operation of said medical device is achieved.

The at least one parameter indicating element may be adjustable relative the connector body such that a position of the at least one parameter indicating element is adjustable to provide a user with a use value associated with the use parameter.

The received signals thus can be used to record or ‘count’ use events associated with the usage of the medical device, i.e. use parameters. In use, the parameter indicating element is simply adjusted or indexed into a new relative position after each event. The resulting unique ‘count’ is retained in a non-volatile manner until the next cycle occurs since the value is based on position and not dependent on power or data

The physical distance associated between the D1 and D2 points is >2 mm and <7 mm.

Since the parameter indicating element length can itself be in the range 2 mm to 9 mm, in some cases the component can travel a distance that is longer than its own length within the connector. This allows for a high resolution of measurement from the individual responses ranging from R1 to R2 for any given parameter indicating element. As shown in the example embodiment of FIG. 5c with at least 5 or more distinct positions and responses. In a preferred embodiment, at least 10 positions are available.

In one embodiment, adjustment from the first discrete position to the second discrete position results in a larger response change than subsequent steps. (FIG. 5c). In one embodiment, the adjustment to an end discrete position from a preceding discrete position may result in a larger response compared to the preceding steps. These differences in responses are advantageous and beneficial as a means of robustly indicating that a critical first or final adjustment has occurred.

Each type of medical device from a plurality of differing medical devices may have a common connector fitted with a different parameter indicating element such that differing identification responses can be achieved. This is shown in the differing response curves shown in FIG. 5a and can also involve varying other parameters such as component material, grade, size, shape to provide a multitude of response ranges and values. The range of responses R1 to R2 can differ in magnitude between different parameter indicating element types for similar positions D1 to D2. Further, different ranges of positions D1 to D2 can be used for different parameter indicating elements. Through the use of these various aspects of the invention, it is possible to construct both a coding scheme to identify a wider range of medical devices and also a means of having a variable value associated with a use parameter (such as used for recording reprocessing cycles).

An example of the use of these techniques involves ensuring certain operational features can be automatically enabled or associated only with detected response values in certain regions of the response curve. For example, to compensate for the change in material characteristics of a medical device, differing control system parameters and operation can be deployed only with the associated usage count of increased distance (e.g. d>>D1) and the corresponding higher response (e.g. r>>R1). This allows for changes to provide improvements in the operation and safety of the medical device to be applied only during the latter parts of the devices operational life when they are specifically required.

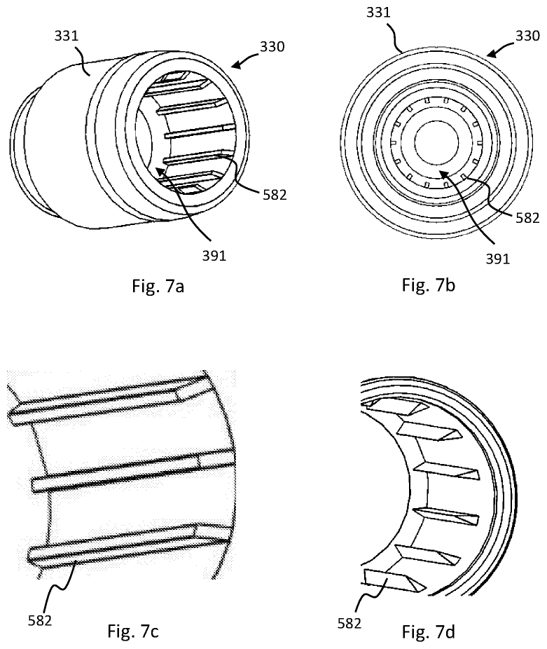

The physical adjustment between positions D1 and D2 can be achieved by providing a screw-like feature so that the position of the identification device is adjusted though rotation—for example using a parameter indicating element with an engagement feature detailed in FIG. 3a-3c. This adjustment means can be combined with improvements in the use of compression ribs for retention of the parameter indicating element in the connector. The use of ribs for retention purposes is further described in the applicant's prior art (e.g. U.S. Pat. No. 10,675,210). This use of ribs is shown in FIG. 7a in perspective, FIG. 7a-b viewed into the connector body, with detailed views in FIG. 7c and FIG. 7d. According to this embodiment, the connector body is connectable to the connecting member by means of insertion into said connecting member.

In one embodiment, the position of the at least one parameter indicating element 390 is arranged to be adjusted in response to the connector 330 engaging the connecting member 310. The at least one parameter indicating element may be arranged to be adjusted in response to the connector 330 engaging the connecting member 310 beyond a threshold engagement distance.

A threshold engagement distance herein refers to a set distance of insertion of either the connector 330 into the connecting member 310 or the connecting member 310 into the connector 330. In one embodiment, said threshold engagement distance may be associated with a full connection between the connector 330 and the connecting member 310.

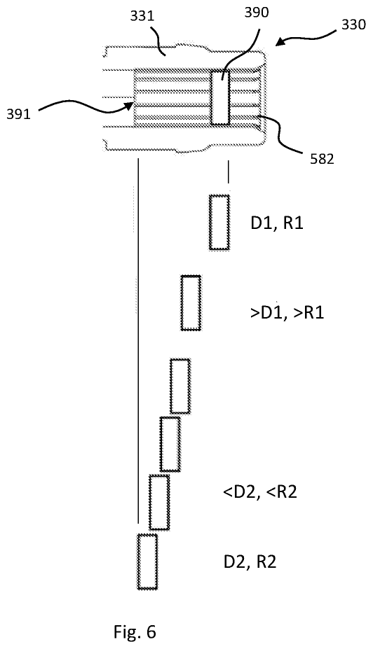

The embodiments described above with reference to FIGS. 1 to 5 and below with reference to FIGS. 6 to 8 are particularly beneficial when the performed use event is performed connections of the connector 330 to the connecting member 310 since the performed connections may manually or automatically adjust the position of the parameter indicating element 390.

Referencing FIG. 6, the connector 330 may comprise retention ribs 582. The retention ribs 582 may protrude from the connector body 331. The retention ribs 582 may be adapted to adjustably retain the parameter indicating element 390 inside the connector body 331. In the prior art, such ribs have not been arranged to allow for any movement of the identification device, thus making them unsuitable for this implementation.

Said retention ribs 582 may be arranged inside the connector body 331 and extend along the length of said connector body 331. The retention ribs 582 extends radially inwards towards a center axis of the connector body 331.

Advantageously, the retention ribs 582 are resilient such that a connection of the connector body 331 to the connecting member causes an adjustment of the position of the parameter indicating element 390. Thereby, an automatic adjustment of the position of the parameter indicating element 390 is achieved.

Preferably, the ribs may be formed during the molding of the connector and are formed from the same material as the connector body, such as low density polyethylene (LDPE) or a similar plastic material.

The retention ribs may comprise a large number (preferably 10-16) of physically separate ribs located around the aperture of the connector body to provide circumferential compression force on the parameter indicating element.

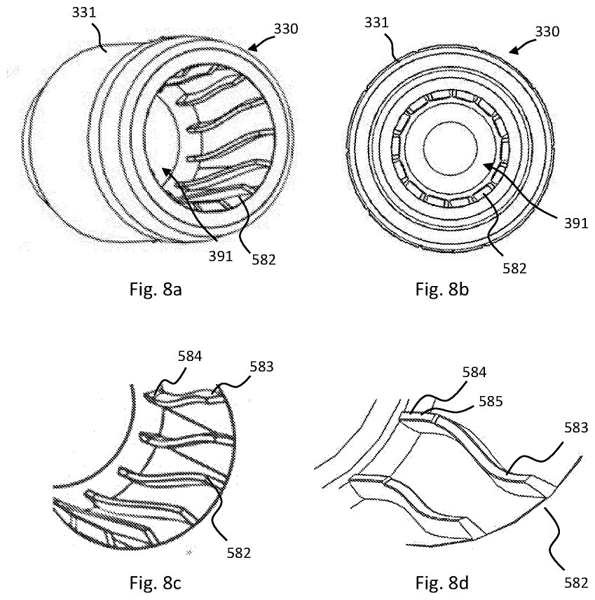

Referencing FIG. 8a-8d, the retention ribs 582 may be arranged in an angle relative the parameter indicating element 390 and the length of the connector body 331 such that adjustment of the parameter indicating element 390 in one direction causes a gradually increasing compressive force for retaining said parameter indicating element 390.

The angled ribs are intended to deform to provide a compression force to the identification device ensuring its retention at a range of positions. As a result, a larger surface of the ribs is in contact with the parameter indicating element surface and able to provide more effective compressive engagement than is the case in the prior art. The resulting arrangement results in a longer rib dimension for any given depth/position than is the case with a straight rib arrangement as in the prior art. This also results in a more effective helical distribution of rib material during insertion ensuring it is provided at different depths of the connector/connecting member interface at an increased range of circumferential positions. The result being an increase in the total area of contact achieved post-insertion of the parameter indicating element with the deformed ribs.

Thus the angled ribs result in a progressive increase in the compressive retention force as the component is moved further into the connector body.

Further, the compressive force adjustably retaining the parameter indicating element 390 may be increased during insertion of the connector body 331 into the connecting member.

In one embodiment, the retention ribs 582 may have a lower height proximal to a distal end of the connector body 331 which first engages the connecting member, whereby the height of the retention ribs 582 progressively increases in a proximal direction of the connector body. This helps with maintaining sufficient retention during the repeated positional adjustments. As the non-compressed rib material is progressively accessed through increasing the depth of insertion of the parameter indicating element 390, the compressive force is maintained and hence the retention characteristics are assured as the rib material deformation process occurs. This is important to ensure that the parameter indicating element 390 is retained throughout the increased lifetime of the device associated with multiple adjustments and associated reprocessing cycles.

In one embodiment, the retention ribs 582 may have a wishbone shape. This wishbone form is such that they are able to provide a torsional support during insertion and adjustment of the parameter indicating element.

The wishbone shape and the material flexibility allows a rotational element to the compressive force holding the parameter indicating element within the barrel. This facilitates the use of a rotational screw motion to achieve positional change without necessarily requiring a screw thread feature on the parameter indicating element outer surface.

In one embodiment, (detailed in FIG. 8c) each rib 582 may be formed from two spline curved portions 583, 584 arranged in an opposing manner in series along its length. This defines the torsional characteristics and ensures multi-directional material deformation during insertion of the parameter indicating element. The spline curved portions 583, 584 may have an angle relative the center axis of the connector body of approximately between 10 and 30 degrees, preferably 20 degrees.

In one embodiment, the retention ribs 582 may be provided with one or more raised portions 585 along the length of the retention ribs (detailed in FIG. 8d). Said raised portions 585 may in one embodiment define the discrete positions of the parameter indicating element 390 relative the connector body 331. The raised portions 585 may be in the form of portions which protrudes a longer distance towards the center axis of the connector body 331 than the remaining retention rib 582.

The raised portions 585 may thus be positioned at key positions along the length to allow the parameter indicating element to be adjusted up to a defined position relative the connector body 331.

Referring back to FIG. 6, one raised portion may be arranged to allow the parameter indicating element to be adjusted up to the limit distance D2.

One raised portion 585 of the retention rib 582 may be provided such that there is an increased mechanical interference with the parameter indicating element 390 to ensure that the parameter indicating element 390 normally remains located in the operational region of ‘C’ as depicted in FIG. 5a.

In one embodiment, the rib may extend beyond the raised portion for a further insertion depth of 2 mm to allow for sufficient travel of the parameter indicating element 390 beyond the normal operation point and to ensure this extreme positioning beyond range ‘C’. This part of the rib can therefore be used to physically set the position of the parameter indicating element 390 beyond the operational range of ‘C’ in FIG. 5a and into the region identified as ‘D’. Thus, if the detected position is indicative of the parameter indicating element 390 being in the region identified as D, an indication may be provided to a user indicating that further use of the product should be prohibited. In one embodiment, the controller device may be configured to render the connector inoperable in response to the parameter indicating element 390 being in the region identified as D. Hence, it is a means of rendering the connector inoperable in combination with a suitably enabled controlling device.

Further referencing FIG. 8a-d the retention ribs 582 may have an modified aspect ratio in terms of the height to base dimension compared to the prior art to further facilitate their function and operation during the fitting and multiple adjustment of the parameter indicating element position. The ratio of height to base of <1 is preferable compared to the ratio of >1 used in in the prior art.

In one embodiment, the connector body 331 may be provided with a circumferential spacing of retention ribs between every 10 and 40 degrees and preferable 25 degrees. This ensures a large contact area between the parameter indicating element and the retention ribs.

According to this embodiment, the parameter indicating element is made of a hard material such as ferrite or brass and has a generally cylindrical or toroidal form, with an outer diameter in the range 6 mm to 9 mm. The length of different component variants ranging from 2 mm to 12 mm. The inner diameter of the parameter indicating element having a diameter of >4 mm to allow for the unobstructed connection through the component. The softer plastic rib material provides a degree of cushioning and dampening for the more brittle ferrite material during repeated insertions and handling.

Table 1 laid out below shows the relationship between parameter indicating element, connector body and associated retention ribs. The rib surface provides an available contact area (prior to material deformation and compression) and the corresponding proportion of parameter indicating element surface area held in contact are compared in Table 1 below. The design metric of % engagement of available rib contact area to parameter indicating element barrel area is calculated. The table shows the improvement achieved in the % engagement as a result of the wishbone shaped rib.

TABLE 1
Comparison of retention rib dimensions

| Parameter indicating element | | | | | | |
|---|---|---|---|---|---|---|
| Dimensions (Examples) | | | | % | | % |
| | | Outer | Straight | engagement | Wishbone | engagement |
| | Outside | Surface | Rib Contact | prior to | Rib Contact | prior to |
| Length | Diameter | Area | Area | compression | Area | compression |
| 3.6 mm | 7.8 mm | 88.2 mm² | 0.61 mm × | 9.6% | 0.94 mm × | 14.9% |
| | | | 14 = 8.54 mm² | | 14 = 13.16 mm² | |
| 7.3 mm | 7.8 mm | 178.8 mm² | 1.23 mm × | 9.6% | 1.90 mm × | 14.9% |
| | | | 14 = 17.22 mm² | | 14 = 26.68 mm² | |

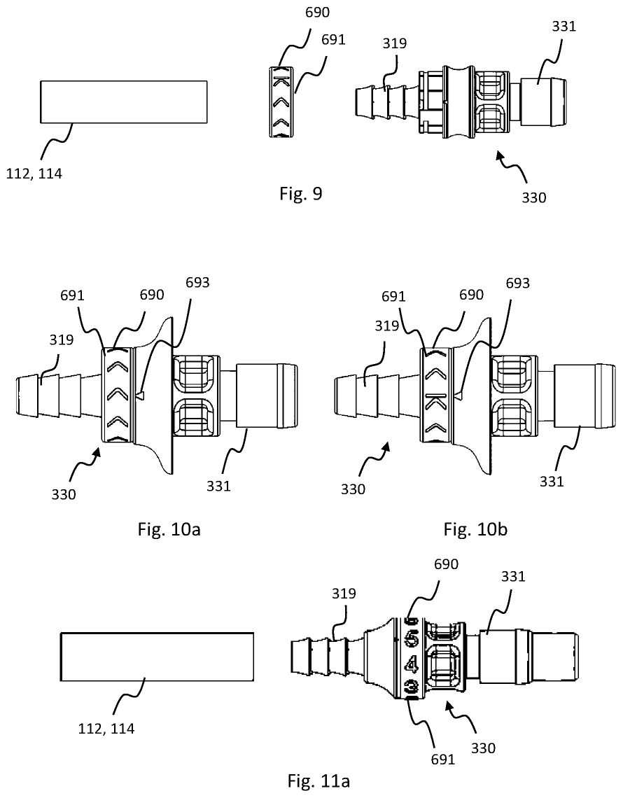

Turning to FIG. 9-13, embodiments wherein one of the at least one parameter indicating elements 690 is adapted to provide a visual and/or tactile indication to user on the connector 330 are depicted.

The parameter indicating element 690 may thus provide a user-accessible mean for providing an indication of a use parameter of the medical device control system. Thus, the position of the parameter indicating element 690 is adapted to directly provide a user an indication of a use parameter.

The parameter indicating element 690 may be provided with means to visually indicate its position and/or use values associated with a use parameter. The user may thus manually adjust the position parameter indicating element 690. For example, the user may manually adjust the parameter indicating element 690 to increment a counter value after a use event has taken place.

In one embodiment, the counter value may be a counter value of reprocessing cycles of a medical device involving any of the steps of assembly, packaging, use, collection, distribution, cleaning, disinfection or sterilization.

In one embodiment, the connector may comprise more than one parameter indicating element 690. In one embodiment, one of the parameter indicating elements may comprise a parameter indicating element providing an indication on the connector and one parameter indicating element which is adjustable so as to be detectable by means of the sensing arrangement as described with reference to FIG. 4-8.

The identification devices may be independently adjustable. In an alternative embodiment, the position of a second parameter indicating element can be directly related to the position of the first parameter indicating element such that adjusting the first parameter indicating element also adjusts the position of the second parameter indicating element. This allows the visual indication provided by means of the controller device and the sensing arrangement to be directly related to the visible indication provided at the connector by means of the first parameter indicating element.

The at least one parameter indicating element 690 may comprise a collar 691. The collar 691 is adjustably mounted to the connector body 331. The collar 691 may be rotatable relative the connector body 331.

In one embodiment, the collar 691 is rotatable relative the connector body 331 so as to be adjustable between a plurality of discrete positions for providing the indication of a use parameter to the user.

In one embodiment, the connector 330 may be provided with visual indicators such as markings, numbers, colors etc. for providing indications of use values and/or discrete positons of the parameter indicating element 690. The visual indicators are located at the connector 330 to be convenient for the point of physical insertion and connection to the medical device control system and may hence be operated by the healthcare worker, clinician or end user in a user friendly manner.

The parameter indicating element 690 may as previously described be adjustable between a plurality of discrete positions. The parameter indicating element 690 may be provided with visual indicators matching the plurality of discrete positions.

Thus, a first discrete position may be indicated by the first visual indicator indicating a use value. Correspondingly, a subsequent discrete position may be indicated by a subsequent visual indicator indicating a use value. A final discrete position may be indicated by a final visual indicator indicating a use value, i.e. a final use value. The final use value may be indicative of the end of acceptable use for one of the components of the medical device control system. The final discrete position may be considered as discrete end position.

Accordingly, the connector 330 may further comprise component retention means. The plurality of discrete positions includes an end position. The component retention means may be arranged to engage the parameter indicating element 390 when the parameter indicating element 390 reaches said end position such that further adjustment of the parameter indicating element 390 is prevented.

This may be achieved by means of compression ribs as described with reference to FIG. 6-8 or by means of one surface being provided with threads, raised step features or other mechanical aspects to allow for travel and a limit to the adjustment of the parameter indicating element.

Referring back to FIG. 8d, the retention means may be in the form of a step 585 arranged to prevent movement of the parameter indicating element 390 beyond a certain point.

In one embodiment, the retention means may be in the form of a retention element such as a slot, ramp or ridge. The retention element may be adapted to prevent movement beyond a certain point when the parameter indicating element has been fitted to the connector body.

In one embodiment, the connector 330 further comprises an auxiliary indication element 691 movable relative the connector body 331. The auxiliary indication element is coupled to one of the at least one parameter indicating elements 690 such that adjustment of the position of said one of the at least one parameter indicating element 690 causes corresponding movement of the auxiliary indication element.

The connector 330 may further comprise an actuation element movable relative the connector body (331), said actuation element being coupled to one of the at least one parameter indicating elements 690 such that movement of said actuation element causes adjustment of the position of said at least one parameter indicating element 690.

In one embodiment, the at least one parameter indicating element 690 is adjustably mounted to the connector body 331 by means of a locking arrangement. The locking arrangement is adapted to permanently secure the at least one parameter indicating element 690 to the connector body 331 upon receiving the at least one parameter indicating element 690.

In one embodiment, the locking arrangement may comprise a one-way ramp provided on the parameter indicating element 690 or the connector body 331. The one-way ramp is arranged to only allow passage in one direction. The one-way ramp may be in a resilient material.

Thus in one embodiment, the locking arrangement is provided on the parameter indicating element 690. In one embodiment, the locking arrangement is provided on the connector body.

The connector 330 may further comprise a tactile indicating device arranged to provide a tactile feedback to a user in response to the at least one parameter indicating element 390 being adjusted between the plurality of discrete positions.

In one embodiment, the tactile indicating device may comprise a ratchet mechanism connecting the parameter indicating element 690 and the connector body 331.

The tactile indicating device may be arranged so that it requires an increased level of force to adjust the position of the parameter indicating element 390 beyond a predefined discrete position of the plurality of discrete positions. This may be readily achieved through the dimensioning of the engagement interface and ratchet mechanism between connector body 331 and the parameter indicating element 690.

In one embodiment, the tactile indicating device may be arranged to have provide lower tactile feedback level for a first set of discrete positions and a higher tactile feedback level for a second set of discrete positions.

In one embodiment, the connector 330 may be provided with a first connection mean for establishing the connection with the connecting member and a second mean for receiving the parameter indicating element. The first connection mean may be in the form of a distal part of the connector adapted to extend into the connecting member.

As previously described, the second connection mean may be in the form of a locking arrangement for permanently securing the parameter indicating element to the connector body 331 while allowing for adjustment of said parameter indicating element relative the connector body 331.

Alternatively, the second connection mean may be in the form a releasable mounting arrangement. This is however less beneficial since it allows for tampering by removal of the parameter indicating element.

The size and dimensions of the engagement means associated with the rotation can be varied to provide differing levels of travel and tactile feedback associated with the rotational position. These levels can also be associated with different regulatory approvals for the number of processing cycles or for the process used. For example, this may be suitable for use with for a device approved for 10 cycles with a first reprocessor using a benign process but the same device may only have approval for 5 reprocessing cycles with a different reprocessor using a more aggressive process which limits device life.

In one embodiment, the medical device may be a sterile device. The use parameter may accordingly be the sterilization state of said medical device. In such an embodiment, there may only be two values which may be provided to the user, i.e. the device being sterilized or non-sterilized. The parameter indicating element may thus be adjustable between a first position indicating a sterilized medical device and a second position indicating a non-sterilized medical device.

In one embodiment, the parameter indicating element when manufactured would be set to the first position to show it is not used, the user on first accessing the device (such as when removing the device from its sterile packaging) then advances the indicator to the second state. This ensures that the connector is capable of visually and permanently indicating that it should not be reprocessed/sterilized in future. It provides a process control check to prevent against the accidental re-preprocessing of sterile devices that are not approved/capable of being returned to a sterile form.

This may also be advantageous where the medical device or medical need is specifically designed for single use in which case the device should never be reprocessed. This is beneficial in the case of true-single use devices such as sterile compression sleeves. The connector can also be fitted with a parameter indicating element that can be used to indicate in conjunction with specific marking or color coding that the product is sterile or single use and hence is not compatible with any reprocessing. This feature is intended to assist a reprocessor with identifying and separating otherwise similar looking devices.

The number of available positions may vary from device to device as needed by the nature of the device and the limitations of its usage. For a compression garment, for example, this could include a number in the range 1 to 50, with preferred embodiments being 2, 5, 6, 10 and 11.

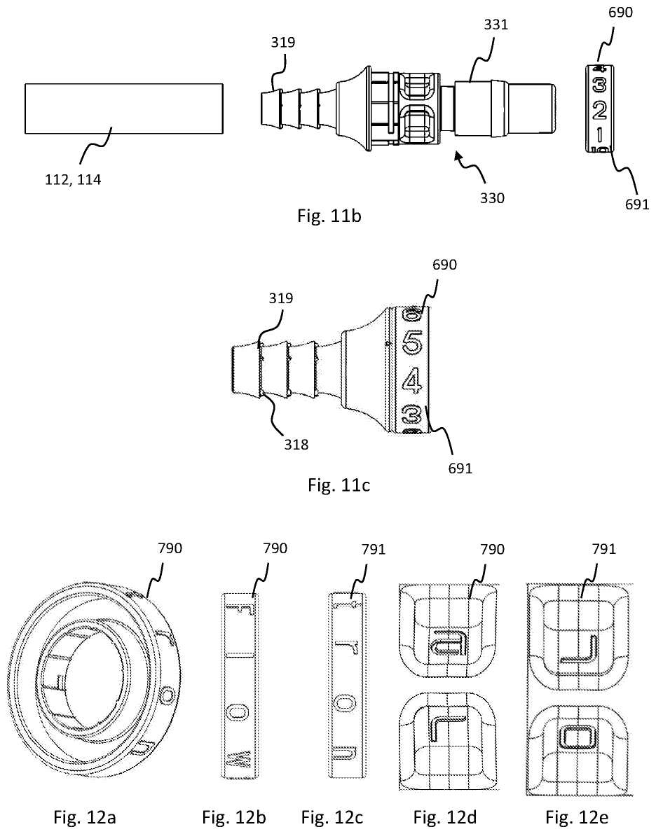

The visual indicators associated with the positions could be indicated numerically (as depicted in for example FIG. 11a-c) or graphically via symbols or characters (as depicted in for example FIG. 9). The indication could be set to either indicate the number of historical cycles of use against an upper limit (e.g. count up usage in a positive manner) or could be set to indicate remaining cycles of use available (e.g. count down available usage). Both of these approaches allow the user to compare the current state against a defined limit and are within the scope of the invention. The cycles of use in this example can relate to uses by different individual patients or can apply to cycles of cleaning/reprocessing. Elements of said visual indicators may for example be provided on the collar 691 and/or the connector body 331.

As depicted in FIG. 10a-b, the connector may comprise marking means 693. The marking may be positioned to either the connector body 331 or the parameter indicating element 690. The marking means may include a number of individual marking elements or a reference pointer 693.

Whilst it is preferable if the visual indications are located on the collar 691 and the marking means are located on the connector body (as shown in FIGS. 9 to 11), it is within the scope of the invention that these locations can be reversed.

In one embodiment, the visual indications are limited to an upper threshold, for example denoted by a numerical representation such as 5 (as shown in FIGS. 11a and 11c) or higher such as 10 or using a specific symbol to represent the upper threshold (as shown in FIG. 10b). An example of the use of a range of numbers covers the numbers 1 to 5 or 1 to 10 or 1 to 20. The indication can be in form of numerals, markers or other graphical symbols that indicate a progression of usage from an initial point to an end point. These indications can be positioned circumferentially on the collar to allow the rotation of the collar to align the markings with the reference pointer on the connector.

In one embodiment, the parameter indicating element 690 may comprise means for receiving the data storage device (as described with reference to FIG. 4). Thus, the parameter indicating element 690 and the data storage device may be mounted to the connector in a single operation. This allows for an easier mounting of the desired functionality to the connector.

With reference to FIG. 9-11, the connector body 331 may comprise a barbed portion 319 for connection with the medical device connection 112 or the controller device connection 114. The barbed portion is arranged to prevent rotation of said medical device connection 112 or controller device connection 114 relative the connector 330.

The barbed portion 319 is arranged to provide an interference fit and a semi-permanent attachment. As a result the connector can not be readily attached or detached from aforementioned connections. It is also preferable if the connection is not able to rotate on the connector and this is achieved using the barbed portion and the addition of teeth-like features included on the barbs for this purpose as shown in FIG. 11c.

The barbed portion 319 may be provided at a proximal end of the connector body 331.

Further referencing FIG. 11c, the connector 330 may comprise a plurality of teeth 318. Said plurality of teeth 318 extends outwardly for engaging the medical device connection (not shown) or the controller device connection (not shown) in an interference fit. The interference fit mitigates the risk for tampering by removal of the parameter indicating element by means of removal of the entire connector. Thereby, a connector which indicates that a component has reached the end of use may not be easily replaced with a new one which indicates that the device is free to use.

The teeth may protrude radially from the connector body 331. The teeth may be in the form of thorns extending outwards from the generally chamfered shape of the barbed portion 319. The barbed portion 319 may be formed by a sequence of chamfered shapes extending in the direction of said barbed portion.

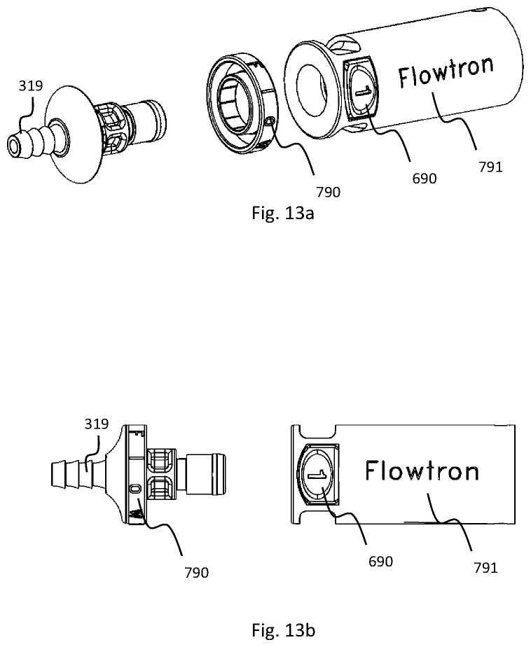

With reference to FIG. 12-13, the connector may be further provided with component identifying means 790. The component identifying means may be fitted to the connector body. Said component identifying means may be adapted to indicate compatibility between the controller device and the medical device. It can be used to demonstrate device compatibility through the use of similar graphics, colors, logos, trademarks and product names.

To ensure compatibility, the controller device or medical device may be provided with similar component identifying means 791.

This aspect helps to ensure that the user combines only suitably compatible parts together and so helps to minimize clinical errors due to the use of otherwise visually similar and mechanically compatible but incorrect combinations. This avoids situations where visually similar devices that are non-approved or non-compatible can physically be combined through misconnection by the user with associated risks to the patient.

In one embodiment of the invention, the component identifying means on a connector for a medical device may provide visual marking of at least one item of information from the list of model number, product type and manufacturer name, brand name, trademark, logo, color or other product-specific feature or aspect.

According to an aspect, a coupling assembly 300 is provided. The coupling assembly 300 is for connecting a medical device 120 and a controller device 110 in a medical device control system 100. The controlled device 110 configured to control the operation of the medical device 120. The coupling assembly 300 comprises a connecting member 310 and a connector 330 according to any one of the described embodiments.

According to an aspect, a medical device system is provided. The medical device system comprises a medical device 120, a medical device connection 112 and a connector 300 according to any one of the described embodiments, the medical device 120 being connectable to the connector 330 by means of the medical device connection 112.

According to an aspect, a medical device control system is provided. The medical control system comprises a medical device 120 and a controller device 110 for controlling the operation of the medical device 120. The medical device control system further comprises a coupling assembly according to any one of the above embodiments.

According to an aspect, a method for providing a user with an indication of a use parameter of a medical device controls system according any one of the above described embodiments is provided.

The method comprises adjusting the position of the at least one parameter indicating element 390, 690 relative the connector body 331 in response to a performed use event associated with the use parameter.

In one embodiment, the method may further comprise detecting the position of the at least one parameter indicating element 390, 690 by means of a sensing arrangement 420 of the medical device control system and providing an indication of the use parameter to a user by means of an indicating device 117 based on a detected position of said at least one parameter indicating element obtained by the sensing arrangement.

In one embodiment, the method may further comprise adjusting the position of the parameter indicating element 390, 690 such that a visual indication of the use parameter is provided to a user on the connector 330.

The invention has been described above in detail with reference to embodiments thereof. However, as is readily understood by those skilled in the art, other embodiments are equally possible within the scope of the present invention, as defined by the appended claims.

1-48. (canceled)

49. A connector for a coupling assembly for connecting a medical device and a controller device in a medical device control system, said controller device being configured to control the operation of the medical device, the connector having a connector body being connectable to a connecting member of the coupling assembly for forming a connection,
wherein the connector comprises at least one parameter indicating element provided on the connector body, said at least one parameter indicating element being adjustable relative the connector body for providing an indication of a use parameter of the medical device control system to a user.

50. The connector according to claim 49, wherein the medical device is an inflatable/deflatable article and the controller device is a pump.

51. The connector according to claim 49, wherein the at least one parameter indicating element is adjustable between a plurality of discrete positions relative the connector body.

52. The connector according to claim 49, wherein the at least one parameter indicating element is adjustable relative the connector body such that the position of the at least one parameter indicating element is adjustable to provide a user with a use value associated with the use parameter, wherein the use value relates to a performed use event of the use parameter.

53. The connector according to claim 52, wherein the performed use event is any one of the group: cleaning of any one of the components of the medical device control system, disinfection of any one of the components of the medical device control system, sterilization of any one of the components of the medical device control system, unpackaging of any one of the components of the medical device control system, assignment of the medical device to a patient, reprocessing of the medical device, uses of any one of the components of the medical device control system or established end of use of any one of the components of the medical device control system.

54. The connector according to claim 52, wherein the use value indicates a counter value and the parameter indicating element is adapted to provide a user an indication in response to the counter value exceeding a predefined counter value, and
wherein the position of the at least one parameter indicating element is arranged to be adjusted in response to the connector engaging the connecting member.

55. The connector according to claim 49, wherein the at least one parameter indicating element is adjustable only in one direction.

56. The connector according to claim 49, further comprising an actuation element movable relative the connector body, said actuation element being coupled to one of the at least one parameter indicating elements such that movement of said actuation element causes adjustment of the position of said at least one parameter indicating element.

57. The connector according to claim 49, wherein the position of one of the at least one parameter indicating element is detectable by means of a sensing arrangement of the medical device control system such that an indication is provided based on a detected position of the parameter indicating element obtained by the sensing arrangement, and
wherein the at least one parameter indicating element is in the form of an identification component, said identification component being adapted to generate a characteristic response associated with any one of the components of the medical device control system, said characteristic response being detectable by means of the sensing arrangement for identifying said one of the components of the medical device control system.

58. The connector according to claim 49, wherein the at least one parameter indicating element comprises any one of ferrite, brass or steel.

59. The connector according to claim 49, wherein the at least one parameter indicating element is arranged inside the connector body.

60. The connector according to claim 49, further comprising retention ribs protruding from the connector body and being adapted to adjustably retain the parameter indicating element inside said connector body, wherein the retention ribs are arranged in an angle relative the parameter indicating element and the length of the connector body such that adjustment of the parameter indicating element in one direction causes a gradually increasing compressive force for retaining said parameter indicating element parameter indicating element.

61. The connector according to claim 49, wherein one of the at least one parameter indicating element is adapted to provide a visual indication to a user on the connector, and
wherein the at least one parameter indicating element comprises a collar adjustably mounted to the connector body.

62. The connector according to claim 51, further comprising:
a tactile indicating device arranged to provide a tactile feedback to a user in response to the at least one parameter indicating element being adjusted between the plurality of discrete positions, and
component retention means,
wherein the plurality of discrete positions includes an end position, and
wherein said component retention means are arranged to engage the parameter indicating element when the parameter indicating element reaches said end position such that further adjustment of the parameter indicating element is prevented.

63. The connector according to claim 49, further comprising a data storage device, said data storage device containing data associated with the medical device or the controller device.

64. A medical device control system comprising a medical device and a controller device configured to control the operation of the medical device, the medical device control system further comprising a coupling assembly for connecting the medical device and the controller device,
wherein the coupling assembly comprises a connector and a connecting member, the connector having a connector body connectable to the connecting member for forming a connection, and
wherein the connector comprises at least one parameter indicating element provided on the connector body, said at least one parameter indicating element being adjustable relative the connector body for providing an indication of a use parameter of the fluid pressure control system to a user.

65. The medical device control system according to claim 64, further comprising:
a sensing arrangement operatively connected to a control unit of the controller device, said sensing arrangement being configured to detect the position of the parameter indicating element, whereby the indication of the use parameter is provided to a user based on a detected position of the parameter indicating element obtained by the sensing arrangement, and
an indicating device operatively connected to the control unit, said indicating device being configured to provide an indication of a use parameter to a user based on the detected position of the parameter indicating element.

66. The medical device control system according to claim 65, wherein the control unit is configured to control the operation of the medical device based on the position of the parameter indicating element.

67. The medical device control system according to claim 65, whereby the at least one parameter indicating element is adjustable relative the connector body such that a position of the at least one parameter indicating element is adjustable to provide a user with a use value associated with the use parameter,
wherein the at least one parameter indicating element is in the form of an identification component, said identification component being configured to generate a characteristic response associated with any one of the components of the medical device control system, the sensing arrangement being configured to detect said characteristic response for identifying said any one of the components of the medical device control system.

68. A method of providing a user with an indication of a use parameter of a medical device control system comprising a medical device and a controller device configured to control the operation of the medical device, the medical device control system further comprising a coupling assembly for connecting the medical device and the controller device, wherein the coupling assembly comprises a connector and a connecting member, the connector having a connector body connectable to the connecting member for forming a connection, and wherein the connector comprises at least one parameter indicating element provided on the connector body, said at least one parameter indicating element being adjustable relative the connector body for providing an indication of a use parameter of the fluid pressure control system to a user, the method comprising:
adjusting the position of the at least one parameter indicating element relative the connector body in response to a performed use event associated with the use parameter.
